(19) United States
(12) Patent Application Publication
(10) Pub. No.: US 2004/0266791 A1
(43) Pub. Date: Dec. 30, 2004

(54) Water soluble salts of risperidone

(76) Inventors: Reinerus G. Gieling, Nijmegen (NL); Desiree Laurant, Nijmegen (NL)

Correspondence Address: MARK R. BUSCHER, P.O. BOX 161, CATHARPIN, VA, 20143, US

(21) Appl. No.: 10/825,684

(22) Filed: Apr. 16, 2004

Related U.S. Application Data
(60) Provisional application No. 60/464,364, filed on Apr. 22, 2003.

(51) Int. Cl. A61K 31/519; C07D 487/04

(52) U.S. Cl. 514/259.41; 544/279

(57) ABSTRACT

Salts of risperidone are provided including solid state salts and water soluble salts. The salts are useful in making pharmaceutical compositions.

This application claims the benefit under 35 U.S.C. § 119(e) from U.S. Provisional Application Ser. No. 60/464,364, filed Apr. 22, 2003, the entire contents of which are incorporated herein by reference.

### BACKGROUND OF THE INVENTION

The present invention relates to salts of risperidone and the use thereof as a pharmaceutical active agent.

Risperidone, or 3-[2-[4-(6-Fluoro-1,2-benzisoxazol-3-yl)-1-piperidinyl]-ethyl]-6,7,8,9-tetrahydro-2-methyl-4-H-pyrido[1,2-a]-pyrimidin-4-one, is a serotonin antagonist approved for the treatment of psychotic disorders such as schizophrenia. Its structure is shown in formula (1).

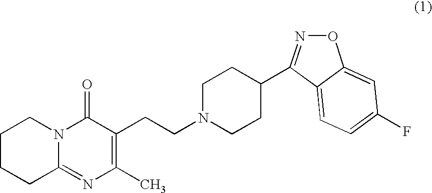

Risperidone is approved for marketing in the U.S.A. under the name RISPERDAL by Janssen, as a free base in both tablet and oral solution dosage forms. Risperidone base is only sparingly soluble in water (approximately 4 mg/ml).

The compound and its pharmaceutical activity are identified in U.S. Pat. No. 4,804,663 as one of several compounds in a class of 3-piperidinyl-1,2-benzisoxazoles or -1,2-benzisothiazoles. Although pharmaceutically acceptable acid addition salts of the entire class of compounds disclosed in U.S. Pat. No. 4,804,663 are taught as being useful, the examples therein synthesize and pharmaceutically test only the free base form of the compounds.

U.S. Pat. No. 5,453,425 and U.S. Pat. No. 5,616,587 disclose stable aqueous solutions of risperidone for oral or parenteral administration. Apparently, the generic solution formulations disclosed in EP 0 196 132, which corresponds to U.S. Pat. No. 4,804,663, provide unsatisfactory stability when risperidone is used as the active ingredient. Both of these patents, U.S. Pat. No. 5,453,425 and U.S. Pat. No. 5,616,587, disclose the use of a buffer to maintain the pH of the aqueous solution within the range of 2 to 6. The solution is taught to be essentially free of sorbitol. The buffer system is described as a mixture of appropriate amounts of an acid and a base. A tartaric acid/sodium hydroxide buffer system is preferred. The solution is taught to be formed by, inter alia, dissolving the acid component of the buffer and the risperidone into heated water; stirring until complete dissolution; and then cooling the solution and adding the base component of the buffer to adjust the pH. The solution can be further diluted with water to a final end-volume.

U.S. Pat. No. 5,616,587 further explains that the tartaric acid/sodium hydroxide buffer system is preferred in part because risperidone tartrate has good aqueous solubility and further reports that risperidone tartrate has a room temperature solubility of about 80 mg/ml while risperidone hydrochloride has a room temperature solubility of about 19.6 mg/ml. However, no description is set forth on how the salt was formed, whether it was formed as a solid and/or isolated form, or on how the solubility test was made. Indeed, given how the solution is formed, it would appear that the salt was formed in situ, i.e. in a dissolved state, and the solubility limit determined from the maximum amount of risperidone base that could be dissolved into the solution. In any event and regardless of such speculation, the patent does not disclose obtaining a solid form of a risperidone salt.

It would be advantageous to provide a pharmaceutically suitable risperidone salt form. It would be further advantageous to provide a stable solid state salt form. Moreover, a water soluble risperidone salt, especially in solid state, would be desirable for a variety of reasons including handling and purification as well as in vitro and in vivo dissolution.

### SUMMARY OF THE INVENTION

The present invention relates to the discovery of water soluble risperidone salts. Accordingly, a first aspect of the invention relates to a salt of risperidone in solid state having a water solubility of at least 10 mg/ml. The salt preferably has a solubility within the range of 20 to 200 mg/ml. The salt is preferably a pharmaceutically acceptable acid addition salt. Typical salt forming acids include hydrochloric acid, methane sulfonic acid, tartaric acid, maleic acid, malic acid, ethane disulfonic acid, lactic acid, acetic acid, and mandelic acid.

Another aspect of the present invention relates to a process for making a solid state water soluble risperidone salt, which comprises contacting a risperidone donor with a suitable acid in a solvent to form a water soluble risperidone salt and precipitating the risperidone salt from the solvent. Preferred solvents are organic solvents including alcohols such as methanol or ethanol and esters such as ethyl acetate.

A further aspect of the invention relates to a salt of risperidone selected from the group consisting of risperidone dihydrochloride, risperidone hydrogenmaleate, risperidone hemitartrate and risperidone hemimalate. The salt may be in solid state or in a dissolved or liquid form.

All of the risperidone salts of the present invention as described above can be used in a pharmaceutical composition in combination with a pharmaceutically acceptable excipient. The composition can be a solid or a liquid form. Further, the risperidone salts of the present invention can be used to treat an animal, preferably a mammal such as a human, by administering an effective anti-psychotic amount thereof to an animal in need of such treatment.

### DETAILED DESCRIPTION OF THE INVENTION

The present invention relates to the discovery of water soluble salts of risperidone. Such salts have a water solubility of at least 10 mg/ml at 20° C. Preferably the water soluble salts exhibit a water solubility of at least 20 mg/ml and typically fall within the range of 20 to 200 mg/ml. For purposes of the present invention, water solubility refers to the solubility in water at about 20° C. The water solubility can be determined by a protocol which comprises stirring a weighed amount of solid risperidone salt with a small amount of water at 20° C., whereby the solid must not dissolve completely (if it does, then a sufficient additional amount of the salt is added in one or more steps until it does not), and measuring the concentration of risperidone in the solution above the solid (=in a supernatant solution). Any suitable method for determining the concentration of risperidone in the solution may be used for purposes of the test (e.g., a gravimetric method based on weighing the solid residue after evaporation of a portion of the solution, a UV-absorption spectrum method, a chromatographic method such as HPLC, etc.). The dissolution of the solid can take a period of time until an equilibrium/steady state between the solid and liquid phases is reached. Thus, if measurements are made directly on the concentration of the supernatant liquid, the measurement are typically repeated until the obtained values of risperidone concentration in the solution are essentially constant. This value is taken as the solubility determination.

A “salt” of risperidone means a mixture of ionic risperidone and counter-ion(s). The risperidone is typically protonated on one or more nitrogen atoms to have one or more positive charges while the counter-ion(s) has one or more off-setting negative charges. The ions can be in a fixed spatial relationship as in a crystal lattice or in an unfixed relationship up to and including a random relationship. Further, the dissolved ions may have some degree of association or the ions can be completely dissociated. Preferably the water soluble salt can be obtained in a solid state. Such a state is useful for handling and/or purification as well as for making a solid state dosage form. The solid state can be crystalline or non-crystalline. When crystalline, it may occur in one or more polymorphic modifications. Further, the solid state form, especially a crystalline form, can be a solvated form, including a hydrated form, or an anhydrous form. Non-crystalline forms include amorphous forms as well as dispersed forms such as molecular dispersions, optionally within a solid matrix material. Non-solid state forms including dissolved forms, i.e., dissolved in a solvent, and oil forms, are also useful in some embodiments of the present invention. The water soluble salts of the present invention can also occur as a mixture of forms such as partly crystalline or as a mixture of states such as solid and dissolved states. Accordingly, water soluble salts of risperidone as used herein embrace all of the above states and forms, unless specifically limited, and are not necessarily in a solid (or necessarily dissolved) state.

The solid state salt is preferably in isolated form; i.e. substantially separated from solvent, such as by filtration or heating, etc., and substantially free from other compounds such as synthetic precursors and/or side products. The solid state salt, whether isolated or not, preferably has a purity of at least 70%, more typically at least 90%, more preferably at least 95%, still more preferably at least 99%, wherein the percentages are based on weight. If intended for use in a pharmaceutical dosage composition, the risperidone salt typically has a purity of at least 99.8% including 99.9%.

In the risperidone salt, the ratio of risperidone ion to counter-ion can vary depending generally upon the counter-ion and the method of formation. This is because risperidone has more than one nitrogen atom that is susceptible to protonation and also many useful acids have more than one proton susceptible of protonating the risperidone base. Hence, risperidone may form various types of salts even with one acid. Generally the molar amount of counter-ion per one mole of risperidone is in the range of 0.5 to 2, but is not limited thereto. Some preferred ratios of risperidone to counter-ion are approximately 1:2 (a “di-salt”), 1:1 (a “mono-salt”), and 2:1 (a “hemi-salt”). Typically variations from these ratios are not greater than 0.1.

Risperidone, having several basic nitrogens, is normally converted to a salt by an acid to make a so-called acid addition salt. Preferably, the risperidone salt is a pharmaceutically acceptable acid addition salt. Suitable acids for making such risperidone acid addition salts include hydrochloric acid, methane sulfonic acid, tartaric acid, maleic acid (cis-butenedioic acid), malic acid (hydroxybutanedioic acid), ethane disulfonic acid, lactic acid, acetic acid, and mandelic acid. Salts made from these acids generally have a water solubility of at least 10 mg/ml. Other acids suitable for making risperidone salts include toluene sulfonic acid, benzene sulfonic acid, naphthalene sulfonic acid, fumaric acid, citric acid, phosphoric acid, and sulfuric acid. However, these salts may not have the desired water solubility and/or may not be pharmaceutically suitable for one or more reasons. Nonetheless, these less preferred salts can still be useful for purification of risperidone, for making water soluble salts of risperidone, or for making pharmaceutical compositions.

The acid addition salts of risperidone include any of the possible molar ratios of risperidone ion to acid ion. For example, the hydrochloric acid addition salt of risperidone includes the mono-salt as well as the di-salt; i.e. risperidone hydrochloride and risperidone dihydrochloride, respectively. While the dihydrochloride salt of risperidone is water soluble, the mono-hydrochloride salt of risperidone has a water solubility of less than 10 mg/ml.

Preferred salt species of the above acids include risperidone dihydrochloride, risperidone mesylate, risperidone hemitartrate, risperidone hydrogenmaleate, risperidone (L)-hemimalate, risperidone hemiedisylate, risperidone (L)-lactate, risperidone acetate monohydrate, and risperidone (R)-mandelate. Each of these salts can be obtained in solid state and exhibits water solubility of at least 10 mg/ml.

Characteristics of various solid state and water soluble salts of risperidone are given in the following table:

| | | solubility (calc. on | |
|---|---|---|---|
| Salt | m.p. (° C.) | free base) | |
| Risperidone dihydrochloride | 285-291 | 84 | mg/ml |
| Risperidone mesylate | 208-212 | >160 | mg/ml |
| Risperidone hemitartrate | 224-227 | 40 | mg/ml |
| Risperidone hydrogenmaleate | 185-186 | 39 | mg/ml |
| Risperidone (L)hemimalate | 183-184 | >300 | mg/ml |
| Risperidone hemiedisylate | 278-283 | 63 | mg/ml |
| Risperidone (L)lactate | 130-133 | 12 | mg/ml |
| Risperidone acetate monohydrate | 161-164 | 72 | mg/ml |
| Risperidone (R)-mandelate | 141-142 | 9.3 | mg/ml |

Other salts that do not exhibit the desired solubility in water but which are also useful in the pharmaceutical industry are listed below.

| | | | |
|---|---|---|---|
| Risperidone tosylate | 220-222° C. | 2 | mg/ml |
| Risperidone napsylate | 188-190° C. | <1 | mg/ml |
| Risperidone Hemifumarate | 214-219° C. | 3 | mg/ml |
| Risperidone besylate | 165-168° C. | 6.5 | mg/ml |
| Risperidone monohydrochloride | 275-279 | 7.3 | mg/ml |

In accordance with the above discussion, it can be seen that some bivalent acids, such as tartaric acid, fumaric acid, edisylic acid or L-malic acid, provide preferably a solid state salt that has 2:1 ratio between risperidone and acid moieties (the “hemi” salts), while some other bivalent acids such as maleic acid preferably provide a solid state salt having the 1:1 ratio (the hydrogen-salts). Thus, as mentioned above, the acid addition salts of risperidone include all possible ratios of acid to base and are not limited to the above preferred species. The acid/base ratios in the formed salt may be determined by a suitable method, e.g. by NMR or by acid titration.

Some risperidone salts may be prepared in solid state in hydrated or solvated forms. For instance, risperidone acetate may be isolated as a monohydrate, risperidone mesylate as a hydrate with variable amounts of water.

The salts of risperidone can be made by contacting risperidone base or a salt thereof (hereinafter collectively a “risperidone donor”) with a suitable salt reaction partner. The contacting typically occurs in a single solution although a mixed phase system can be employed such as a solid-liquid slurry, etc., wherein one or more reactants is not fully soluble in the liquid phase. A suitable salt reaction partner is one that is sufficiently reactive to react with the risperidone donor to form a salt. Preferably the salt reaction partner is an acid, especially a pharmaceutically acceptable acid including those acids specifically recited previously herein.

For making a solid state salt, the process comprises contacting a risperidone donor with a suitable acid in a solvent to form a risperidone salt and precipitating the risperidone salt from the solvent. A suitable acid is one that allows the salt reaction to go forward. For example, if the risperidone donor is itself a salt, then the acid must be sufficiently strong to replace the initial salt anion, as is well known by workers skilled in the art. When the process is used to make water soluble risperidone salts, then a “suitable acid” additionally means that the acid is one that can result in a risperidone salt having water solubility of at least 10 mg/ml.

The amount of the acid used in the process of making risperidone salt is not particularly limited but should advantageously be at least an equivalent amount. For example, for a di-salt at least two moles of acid for each mole of risperidone donor should be provided. While less than an equivalent amount of acid can be used, a slight or even substantial excess of the acid is normally preferred. The “equivalent” may relate to one or more basic nitrogens in the risperidone molecule that are able to be neutralized by the acid. The amount of acid, especially with multivalent acids such as sulfuric acid, phosphoric acid, maleic acid, fumaric acid, tartaric acid, and citric acid, etc., can affect the type of salt formed; i.e. a hemi-salt or a di-salt, etc. For example, risperidone L-tartrate may be isolated in various forms. The hemi-salt is obtainable, for example, by treating risperidone in ethanol with up to 2 equivalents of tartaric acid. Under a higher excess of tartaric acid such as 3 equivalents or more, a tartrate salt may be formed with a molar ratio between risperidone and tartaric acid of 2:3 or even 3:5. Salts comprising qualitatively the same acid anion, but quantitatively different amounts thereof, may exhibit differences in aqueous solubility as well as other properties.

Furthermore, the excess of the acid in the reaction mixture may have an influence on the solubility of the formed salt in the solvent system; it may either increase or decrease the solubility of the formed salt. The amount of the acid in the system may have influence on the morphology of the solid salt that separates from the solvent, i.e. the salt may separate in various polymorphic modifications differing by solid state properties, including solubility in water.

The order and rate of contacting risperidone donor with the acid can vary. Advantageously, the acid, used as such or dissolved or suspended in a solvent, is added at once, portionwise or continually, to a stirred solution or suspension of risperidone donor especially risperidone base. The order of contacting may also be reversed. Preferably, the concentration of risperidone donor, the kind of solvent, and the temperature of contact are so selected that a clear solution of the risperidone salt is, at least temporarily, formed. In any event and regardless of whether slurries or suspensions are employed of the risperidone donor or acid, the salt forming reaction (neutralization reaction) occurs in a dissolved state. The temperature of the solvent during the contact can be constant or variable and is not particularly limited. Typically the solvent temperature is from room temperature (20° C.) up to the reflux temperature of the solvent, and preferably is at least 30° C.

The “solvent” can be a single liquid or a mixture of two or more and thus the term “solvent” embraces the singular as well as the plural forms of the word; i.e. solvents. When the risperidone salt is water soluble, the solvent is preferably a primarily organic solvent wherein water can be present in minor amounts, i.e., not greater than 50%. Generally the solvent comprises a lower aliphatic alcohol, a lower aliphatic ketone such as acetone, an ether such as diethylether or tetrahydrofuran, or a hydrocarbon such as hexane, and mixtures thereof. Preferably the solvent is comprised in whole or in part of a lower aliphatic alcohol (C₁-C₄ alcohols), most preferably ethanol, because they dissolve risperidone base in a suitable extent and they are also able to dissolve a lot of acids that are used for making the salts. Additionally, and advantageously, many water soluble risperidone salts were found to be sufficiently insoluble in the alcohol solvent that the formed salt can be easily isolated in the solid state. If the solubility of the risperidone base or the acid in the alcohol solvent is found to be insufficient for the intended purpose, it may be enhanced by common means, e.g. by heating the mixture (optionally up to reflux) or adding a co-solvent enhancing the solubility.

In a preferred case, the salt, which is formed after contacting the risperidone donor with the acid in the solvent, precipitates from the solution spontaneously due to a difference in solubility between the formed salt and the starting materials in the solvent. Optionally, the precipitation may be induced by a suitable conventional technique(s), or the yield of precipitation may be enhanced by such technique(s). The techniques preferably comprise, alone or in combination:

a) cooling the reaction mixture, including spontaneous cooling, i.e. without applying a cooling device, of a previously heated solution;

b) concentrating the reaction mixture including essentially evaporating/removing the whole amount of the solvent;

c) adding a contrasolvent wherein the contrasolvent—a liquid in which the formed salt is less soluble—may be miscible or immiscible with the solvent; and/or

d) adding a seed crystal at anytime during the process including from before contacting to after precipitation has begun.

In a general process, approximately 30 ml of a C₁-C₄ aliphatic alcohol, e.g. methanol, ethanol or isopropanol, is used per each 1 gram of risperidone base in the risperidone donor, and an equivalent of the corresponding acid is added to the stirred suspension under heating, giving essentially a clear solution. After a short period of stirring, a solid comprising the risperidone salt precipitates either spontaneously or after addition of a contrasolvent, e.g. diethyl ether. In a few cases, it may be necessary to cool the solution on an ice bath, or to reduce the solution's volume. The obtained solid, generally crystals, is then filtered off, washed with ethanol and dried, preferably in vacuo. This general process may also be used in industrial manufacture of the risperidone salts.

In some cases, it can be advantageous to vary from the general process. For instance, an acetate salt of risperidone is preferably formed by contacting risperidone base with acetic acid in a hexane/acetone mixture. Upon addition of water, the risperidone acetate crystallizes as a monohydrate.

After the risperidone salt is precipitated it can be isolated by known techniques such as filtration. Isolated risperidone salt may contain some impurities and may be purified into the desired degree of purity by various methods. For instance, it may be recrystallized from a suitable solvent, preferably a non-aqueous solvent, optionally after treatment with a suitable adsorption material, e.g. with activated charcoal. Suitable solvents include methanol, ethanol, isopropanol, acetone, diethyl ether, tetrahydrofuran, ethyl acetate, and mixtures thereof.

In a particular modification of the salt-forming process, risperidone base and the corresponding acid may be dissolved in liquid carbon dioxide under pressure (supercritical carbon dioxide), at temperatures close to ambient. The solutions are then evaporated by simply decreasing the pressure to ambient. The solid salt is thus formed at ambient temperature, free from any solvent without drying.

Risperidone salts of the present invention are also useful for making other risperidone salts. The inter-conversion may be direct, i.e. a solution of risperidone salt is treated with the corresponding acid, and the desired risperidone salt of such acid precipitates due to its different solubility in the system. This is useful for making non-water soluble salts of risperidone, such as risperidone tosylate, wherein water can be used as the solvent or co-solvent. Alternatively, risperidone salts may be prepared by an indirect two step process, wherein risperidone base is prepared from a salt in a first step, preferably in aqueous medium, via neutralization with a base, and, optionally after isolation thereof, converted in a second step into a salt by treatment with the corresponding acid, preferably in a non-aqueous medium, i.e. an organic solvent(s). This approach is useful for forming water soluble salts of risperidone.

Risperidone salts are also useful for purifying risperidone base by a base-salt-base conversion. Specifically risperidone salt converts into risperidone base by treatment with a stronger base. Thus, the risperidone base may be converted into a salt in a non-aqueous solvent, as described above, while the salt may be advantageously converted back to the risperidone base in an aqueous medium with the addition of base, as risperidone base is sparingly soluble in such medium and may precipitate therefrom. Consequently, using two different kinds of media, one may remove both hydrophilic and lipophilic impurities from the crude risperidone base. Suitable bases for converting the salt into risperidone free base include alkali metal hydroxides such as sodium hydroxide and potassium hydroxide.

Similarly, the principle of salt-base-salt conversion may also be used for the purification of risperidone salts. Using two kinds of solvents allows the removal of various kinds of impurities. Optionally, the solution of risperidone salt in the solvent system is further purified prior to contacting it with the base; it may be extracted by an immiscible liquid or treated with an adsorption material.

A pharmaceutically useful salt of risperidone in addition to having good water solubility, should preferably be easily obtained in isolated form such as by precipitation/crystallization and in high yield. The salt should also provide good thermal stability. Another important property is hygroscopicity in as much as water absorption should preferably be minimal. However, a stable hydrated state is generally acceptable. Last but not least, the salt should have acceptable pH in solution and should be compatible with pharmaceutical excipients. Taking into account a combination of these and other properties, the risperidone salt is preferably risperidone dihydrochloride, risperidone hydrogenmaleate, risperidone hemitartrate or risperidone hemimalate.

Regarding the dihydrochloride salt of risperidone, the above process, wherein two equivalents of hydrochloric acid reacts with risperidone base in ethanol, produces a crystalline risperidone dihydrochloride after spontaneous crystallization. The material may be obtained as an anhydrate. In the absence of drying, or upon standing at ambient temperature in open air, the material may comprise small amounts of absorbed or adsorbed water (less than 0.5%), but is not hygroscopic. The product exhibits higher aqueous solubility (>80 mg/ml) than as reported in WO 96/01652 for risperidone hydrochloride (about 20 mg/ml).

Risperidone dihydrochloride can also be formed by dissolution of risperidone base in ethanol and treatment with concentrated (12N) aqueous HCl, wherein the solid state form of risperidone dihydrochloride is obtained after spontaneous crystallization.

In contrast, treating risperidone base in ethanol with a molar equivalent of aqueous HCl, results in a solid state form of risperidone monohydrochloride slowly crystallizing from the solution. This form has different properties than the above dihydrochloride. The salt may be isolated in hydrated forms or as an anhydrate. This solid state form exhibits lower water solubility (anhydrate form 7.3 mg/ml, calculated as free base).

The risperidone salts of the present invention can be formulated into various pharmaceutical compositions. A suitable pharmaceutical composition comprises a risperidone salt and a pharmaceutically acceptable excipient(s). The pharmaceutical compositions of the present invention include the unit dosage form as well as the intermediate bulk formulations such as pellets, beads, granules, powder blends, concentrated solutions, etc. Typically the composition is a finished dosage form also referred to as a unit dose. Dosage forms include oral dosage forms, topical dosage forms such as a transdermal patch, parenteral dosage forms such as an injectable solution, and rectal dosage forms such as a suppository, but is not limited thereto. Oral dosage forms are the most preferred due to the ease of administration and include solid oral dosage forms such as capsules, tablets, sachets/granules, and powders, as well as liquid oral dosage forms such as solutions, suspensions, and emulsions, most preferably a solution especially an aqueous solution.

The risperidone salt to be used in the pharmaceutical composition can be any salt of risperidone as described above. Preferably a pharmaceutically acceptable acid addition salt of risperidone is used and more preferably a water soluble salt of risperidone is used, but such is not required.

Pharmaceutically acceptable excipients can be in solid state or liquid state as is well known in the art and include carriers, diluents, fillers, binders, lubricants, disintegrants, glidants, colorants, pigments, taste masking agents, sweeteners, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, sugars, and solvents. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; polyalkylene glycols such as polyethylene glycol and polypropylene glycol; and polyacrylic acids including their copolymers and crosslinked polymers thereof, i.e. Carbopol® (B.F. Goodrich), Eudragit® (Rohm), polycarbophil and chitosan polymers. Waxes include white beeswax, microcrystalline wax, carnauba wax, hydrogenated castor oil, glyceryl behenate, glycerylpalmito stearate, saturated polyglycolyzed glycerate. Calcium phosphates include dibasic calcium phosphate, anhydrous dibasic calcium phosphate, and tribasic calcium phosphate. Sugars include simple sugars such as lactose, maltose, mannitol, fructose, sorbitol, sacarose, xylitol, isomaltose, and glucose as well as complex sugars (polysaccharides) such as maltodextrin, amylodextrin, starches, and modified starches. Solvents are typically water or ethanol or a mixture thereof.

Solid compositions for oral administration of risperidone salts may exhibit immediate or extended release of the active substance from the composition. Such compositions preferably comprise a water soluble salt of risperidone and at least one solid state excipient. Solid pharmaceutical compositions are preferably formulated into tablets. The tablets may be disintegrable or monolithic. The tablets may be produced by any standard tabletting technique, e.g. by wet granulation, dry granulation or direct compression. One preferred tablet is an orally disintegrable tablet, i.e. a composition that disintegrates directly in the mouth. Various systems are known in the art and they are applicable to the salts of the invention. Preferred however is an orally disintegrating tablet comprising at least 50% silicified microcrystalline cellulose as described in commonly-owned U.S. Provisional patent application 60/463,027, filed Apr. 16, 2003, the entire contents of which are incorporated herein by reference. The silicified microcrystalline cellulose is preferably the intimate physical mixture of colloidal silicon dioxide with microcrystalline cellulose as described in U.S. Pat. No. 5,585,115. The amount of silicon dioxide is normally within the range of 0.1 to 20 wt % and more typically 1.25 to 5 wt % such as about 2 wt %. Surprisingly, such an excipient can form a tablet matrix that is orally disintegrating; i.e., the tablet disintegrates in the mouth in 80 seconds or less, preferably 2 to 50 seconds. The amount of silicified microcrystalline cellulose is preferably 50% to 90%, more preferably 60% to 80% based on the weight of the tablet.

Risperidone salts may alternatively be blended into compositions that are suitable for being formulated into pellets. A plurality of risperidone pellets comprising the single dose of risperidone may be encapsulated into capsules made from pharmaceutically acceptable material, such as hard gelatin. In another mode, a plurality of pellets may be compressed together with suitable binders and disintegrants to form a disintegrable tablet that, upon ingestion, decomposes and releases the pellets. In yet another mode, the plurality of pellets may be filled into a sachet.

Pharmaceutical compositions comprising risperidone salts and intended as final dosage forms for administration preferably contain a therapeutically effective amount of risperidone. The amount of the risperidone salt, expressed in terms of risperidone base, in the unit dose is usually from 0.1 to 20 mg, preferably 0.25 mg, 0.5 mg, 1 mg, 2 mg, 3 mg, 4 mg, 6 mg, or 8 mg. The unit dose in a tablet form can be one or more tablets administered at the same time. In the last case, several smaller tablets may be advantageously filled into a gelatin capsule to form a unit dose. The unit dose of a granulate or pellets in a capsule form advantageously comprises a single capsule.

Water soluble risperidone salts are particularly suitable for making liquid pharmaceutical compositions for oral or parenteral administration. Preferably these solutions are aqueous, meaning that water comprises a portion of the solvent medium. Usually water comprises at least 50% of the solvent, preferably at least 60%, more preferably at least 80%, still more preferably at least 90%, and most preferably essentially 100% of the solvent. The remainder of the solvent may be, for instance, ethanol. In addition to containing the risperidone salt as an active ingredient and a solvent, these compositions may contain auxiliary ingredients such as preservatives, tensides, isotonizing agents, flavors, colors etc. If necessary, a pH value of the solution may be adjusted by titrating with a suitable acid or base to a desired value. However, it is an advantage of the present invention that the risperidone solution is not required to contain a buffering system. That is, the inventive solution preferably has only a stoichiometric or near stoichiometric amount of acid anion as opposed to a buffer system which requires a molar excess of an acid. Note that stoichiometric in this context means the native ratio of the acid in the salt. For example, a stoichiometric amount of a hemi salt would have approximately a 1:2 ratio of acid to risperidone. Thus, a water soluble salt of risperidone, such as risperidone hemitartrate, could be dissolved in water, or an aqueous solution, without adding additional acid. This is possible because (1) the water soluble salts of risperidone are sufficiently soluble and stable that a buffering system is not needed and (2) many of the salts have a desired pH for making a solution. Solutions of various risperidone salts have the following pH values (at a concentration of 1 mg/ml, calculated on risperidone base):

| | |
|---|---|
| Hemitartrate | 5.17 |
| Hydrogenmaleate | 4.85 |
| Mesylate | 3.10 |
| Hemimalate | 6.40 |
| Dihydrochloride | 3.23 |
| Acetate monohydrate | 6.67 |

Orally administrable solutions should preferably have a pH of 3.5-8.5, while parenterally administrable solutions should preferably have a pH of 4-9. Thus, taking also conventional excipients in account, preferred water soluble risperidone salts are the salts which provide, after dilution with water, a pH of between 3 and 6.5.

Furthermore, the preferred soluble risperidone salts are compatible in solution with carbohydrates/sweeteners such as sorbitol, which is surprising due to the earlier disclosure in WO 96/01652 that sorbitol causes instability of risperidone solutions and should be avoided from the pharmaceutical composition. In testing 1 mg/ml aqueous solutions of risperidone dihydrochloride, hemimalate and hydrogenmaleate comprising 2% sorbitol and 0.2% of benzoic acid at various stress temperatures (up to 80° C.), it was found that such solutions are surprisingly stable.

The liquid dosage forms can be made by conventional and/or simple techniques known in the art. For example, the water soluble risperidone salt can be dissolved into an aqueous solvent before or after addition of any auxiliary ingredients generally with stirring, optionally at elevated temperatures. The process of making the liquid dosage form can be easier and more convenient than solubilizing risperidone free base into an aqueous solution. The liquid composition can be made initially as a concentrated solution, or suspension, and then diluted to a solution in the finished dosage form strength.

The unit dose of an injectable solution is advantageously one vial. Oral solution is preferably delivered in a multidose package, wherein the unit dose may be defined by the number of droplets, teaspoons or by means of a calibrated vial. Preferred concentration of risperidone in oral or parenteral solutions is from 0.1 mg/ml to 10 mg/ml, particularly of about 1 mg/ml or 2 mg/ml.

The risperidone salts can be used to treat psychotic disorders including schizophrenia in animals, preferably mammals such as humans. The method comprises administering an anti-psychotic effective amount of a risperidone salt, especially water soluble risperidone salt, to an animal patient, preferably a mammalian patient, in need thereof. The effective amount is generally within the range of 0.001 mg/kg to 0.4 mg/kg of body weight, more preferably 0.004 mg/kg to 0.2 mg/kg of body weight. Preferably the risperidone salt is administered as a unit dosage from as described above. It should be understood that a single administration includes taking one or more unit dosage forms at essentially the same time, e.g. taking two tablets.

The entire disclosure in each of the patents and journal articles mentioned in the above description is incorporated herein by reference. The invention will be further described with reference to the following non-limiting examples.

### EXAMPLE 1

### Risperidone Dihydrochloride

| | |
|---|---|
| 1.98 g | Risperidone base was suspended in |
| 50 ml | ethanol, affording a white suspension. |
| 2 ml | of an HCl solution (5-6 N in i-propanol) was added, |
| | resulting in an almost clear solution. After one minute, |
| | the solution changed into a milky white suspension. Then, |
| | the suspension was filtered off, washed twice with ethanol |
| | and dried in vacuo at 40° C., affording |
| 1.95 g | risperidone dihydrochloride (91%) |
| | water content: 0.22% |
| | NMR: confirmed the structure |
| | Acid titration: confirmed the di-salt |

### EXAMPLE 1A

### Risperidone Dihydrochloride

| | |
|---|---|
| 1.98 g | Risperidone base was suspended in |
| 30 ml | ethanol, affording a white suspension and heated |
| | until a clear solution was obtained. |
| 1 ml | concentrated HCl solution (12 M; 2.5 equiv.) was added. A |
| | white precipitate was formed immediately. The mixture was |
| | then further stirred for 20 h at room temperature. The precipitate |
| | was filtered off, washed once with ethanol and dried in vacuo at |
| | 40° C. for 5 days, affording |
| 2.03 g | risperidone dihydrochloride (87%), mp. 285-291° C. |
| | water content: 0.16% |
| | water content after exposure to air under ambient conditions for |
| | 3 weeks: 0.30% |
| | Acid titration: confirms dihydrochloride salt |

### EXAMPLE 2

### Risperidone Hydrogenmaleate

| | |
|---|---|
| 0.99 g | risperidone was suspended in |
| 30 ml | ethanol, affording a white suspension. |
| 279 mg | maleic acid was added, resulting in a pale yellow clear |
| | solution. After a few minutes a white solid precipitated. The |
| | precipitate was filtered off, washed with cold ethanol and |
| | dried in vacuo at 40° C., affording |
| 1.04 g | risperidone hydrogenmaleate (82%) |
| | mp. 190-195° C. |
| | NMR: confirmed the structure |

### EXAMPLE 3

### Risperidone Hydrogenmaleate

| | |
|---|---|
| 14.00 g | Risperidone base was stirred with |
| 250 ml | ethanol and heated to 60° C., affording a clear |
| | solution. A solution of |
| 4.00 g | maleic acid in |
| 25 ml | ethanol was added in 5 minutes. The solution was allowed |
| | to cool to room temperature. Crystallization was induced by |
| | seeding at 40° C. After further stirring at room |
| | temperature for 21 h, the crystals were filtered off, washed |
| | twice with ethanol and dried in vacuo at 40° C. for 3 days, |
| | affording |
| 15.65 g | risperidone hydrogenmaleate (87%), mp. 185-186° C. |
| | water content: <0.1% |

### EXAMPLE 4

### Risperidone Mesylate

| | |
|---|---|
| 1.98 g | risperidone was suspended in |
| 50 ml | ethanol, affording a white suspension and heated |
| | until a clear solution was obtained |
| 0.7 ml | methanesulfonic acid was added and stirred for 22 h. |
| | The solution was concentrated at reduced pressure, |
| | affording a yellow oil. Diethyl ether was added, giving rise to a |
| | white precipitate. The crystals were filtered off and dried in |
| | vacuo at 40° C. for 3 days, affording |
| 2.14 g | risperidone mesylate (yield: 87%), mp 208-212° C. |
| | water content: 0.41% |
| | water content after exposure to air under ambient conditions |
| | for 2 weeks: 1.38% |
| | water content after exposure to 40° C./75% humidity for |
| | 2 weeks: 8.51% |
| | Drying experiment with the hydrated product: Water is released |
| | starting from 30° C., yielding the anhydrate. |

### EXAMPLE 5

### Risperidone Hemitartrate

| | |
|---|---|
| 1.98 g | risperidone was dissolved by heating in |
| 30 ml | ethanol. A solution of |
| 1.45 g | (L)-tartaric acid in |
| 20 ml | ethanol was added, resulting in a pale yellow |
| | clear solution, which was allowed to cool to room temperature. |
| | After a few minutes, a precipitation was formed. After stirring |
| | at room temperature for 20 hours, the precipitate was filtered |
| | off, washed once with ethanol and dried in vacuo at 40° C. |
| | for 3 days, affording |
| 1.85 g | risperidone hemitartrate (79%), mp. 224-227° C. |
| | NMR: confirmed the structure |
| | Water content: 0.28% |

### EXAMPLE 6

### Risperidone Acetate Monohydrate

| | |
|---|---|
| 5.00 g | of risperidone base was suspended in a mixture of |
| 15 ml | of n-hexane, |
| 1.5 ml | of acetone and |
| 0.22 ml | of water. Under stirring, |
| 0.73 g | of acetic acid was added dropwise. The paste formed |
| | in the solution was triturated and after several minutes it |
| | turned to crystals. The suspension was stirred for 1 hour at |
| | room temperature. The crystals were filtered off and washed |
| | with 2 × 5 ml of n-hexane. Product was air dried at room |
| | temperature to constant weight. |
| | Yield: 5.25 g, mp. 161.5-164.5° C. |
| | Water content (by K. Fischer): correspond to monohydrate. |

### EXAMPLE 7

### Risperidone Hemimalate

| | |
|---|---|
| 0.99 g | of risperidone was suspended in |
| 30 ml | ethanol, affording a white suspension. |
| 323 mg | (L)-(−)-malic acid was added and after short heating, |
| | a pale yellow clear solution was obtained. The solution was |
| | allowed to cool to room temperature, resulting in crystallisation. |
| | The crystals were filtered off, washed once with ethanol and |
| | dried in vacuo at 40° C., affording |
| 582 mg | risperidone hemimalate (51%), mp. 183-185° C. |
| | NMR: confirmed the structure |
| | Water: 0.9% |

### EXAMPLE 7A

### Risperidone Hemimalate

| | |
|---|---|
| 14.10 g | risperidone was stirred with |
| 250 ml | ethanol and heated to 63° C. in 20 minutes affording |
| | a clear solution. A solution of |
| 2.32 g | (L)-malic acid in |
| 25 ml | ethanol was added in 10 minutes. The solution was allowed |
| | to cool to room temperature in 105 minutes. Crystallization |
| | was induced by scratching in the flask at 25° C. After |
| | further stirring at room temperature for 19 h, the crystals |
| | were filtered off, washed twice with ethanol and dried in |
| | vacuo at 40° C. for 24 h, affording |
| 14.11 g | risperidone hemimalate (86%), mp. 183-184° C. |
| | Water content: 0.40% |

### EXAMPLE 8

### Pharmaceutical Solutions

| | |
|---|---|
| Composition | (m/V %) |

a) Oral solution

| | |
|---|---|
| Risperidone salt (as a base) | 0.1% |
| benzoic acid | 0.2% |
| saccharin | 0.1% |
| NaOH | a.d. pH |
| Flavors | qs |
| Water | a.d. 100% |

b) Parenteral solution

| | |
|---|---|
| Risperidone salt (as a base) | 0.2% |
| NaCl | 0.9% |
| Na EDTA | 0.01% |
| HCl/NaOH | a.d. pH |
| Water | ad 100% |

Preparation of Solutions:

All excipients, starting with risperidone salt, are dissolved in 80% of the quantity of water. After everything is dissolved, the pH is checked and, optionally, NaOH and/or HCl is used to titrate the solutions to the target pH. Finally the solution is brought to its target volume with purified water, resulting in an oral solution of 1 mg/ml or a parenteral solution of 2 mg/ml.

### EXAMPLE 9

### Pharmaceutical Tablets

Composition of tablet mass (per tablet, in mg):

| | Strength (mg of risperidone base) | | | | | | | |
|---|---|---|---|---|---|---|---|---|
| | 0.25 | 0.5 | 1 | 2 | 3 | 4 | 6 | 8 |
| Risperidone (as | 0.25 | 0.5 | 1 | 2 | 3 | 4 | 6 | 8 |
| salt): | | | | | | | | |
| Lactose | 45.5 | 91.0 | 131.0 | 130.0 | 195.0 | 260.0 | 114.6 | 152.8 |
| monohydrate* | | | | | | | | |
| Microcrystalline | 7.5 | 15.0 | 20.0 | 20.0 | 30.0 | 40.0 | 18.0 | 24.0 |
| cellulose* | | | | | | | | |
| HPMC | — | — | 2.0 | 2.0 | 3.0 | 4.0 | — | — |
| Sodium lauryl | 0.15 | 0.3 | 0.4 | 0.4 | 0.6 | 0.8 | 0.36 | 0.48 |
| sulfate | | | | | | | | |
| Pregelatinized | 19.5 | 39.0 | 44.0 | 44.0 | 66.0 | 88.0 | 39.6 | 52.8 |
| starch | | | | | | | | |
| Colloidal silica | 0.225 | 0.45 | 0.6 | 0.6 | 0.9 | 1.2 | 0.54 | 0.72 |
| Magnesium stearate | 0.375 | 0.75 | 1.0 | 1.0 | 1.5 | 2.0 | 0.9 | 1.2 |
| Tablet mass | 76.6 | 153.5 | 200 | 200 | 300 | 400 | 180 | 240 |

The * indicates that the amount of lactose and/or microcrystalline cellulose can be adjusted to compensate for (offset) the differing weights of the risperidone salts so that the tablet composition equals the total targeted mass.

Risperidone salt is mixed well with the cellulose. Then pregelatinized starch is added and mixed. Subsequently, the remaining excipients (except Mg stearate and silica) are mixed and the silica is then added. The entire blend is screened over a 850 micrometer sieve, mixed again, and then the Mg stearate is added. The blend is mixed resulting in a blend for tablet compression. The blend is compressed into tablets comprising 0.25 to 8 mg of risperidone respectively.

### EXAMPLE 10

### Coated Tablets Comprising Risperidone Salt

Coating composition:

| | |
|---|---|
| HPMC | 49% |
| PEG 400 | 12% |
| Titanium dioxide | 24% |
| talc | 15% |
| Colours | qs |

Preparation of Coating Suspension:

All excipients are, one by one, added to water. The suspension is mixed well until a homogeneous mixture is obtained, resulting in a coating suspension containing 10-18% solids.

The 2 mg strength risperidone tablet made in example 9 is coated using the suspension in a coating apparatus. Alternatively, commercially available coating suspensions such as various grades of Opadry® may be used.

### EXAMPLE 11

### Capsules Comprising Risperidone Salts

Composition (per capsule, in mg):

| | Strength (mg of risperidone base) | | | | | |
|---|---|---|---|---|---|---|
| | 1 | 2 | 3 | 4 | 6 | 8 |
| Risperidone (as salt) | 1.0 | 2.0 | 3.0 | 4.0 | 6.0 | 8.0 |
| Lactose monohydrate* | 94.7 | 94.2 | 141.3 | 188.4 | 68.4 | 91.2 |
| Microcrystalline | 94.7 | 94.2 | 141.3 | 188.4 | 86.4 | 91.2 |
| cellulose* | | | | | | |
| Sodium starch | 8.0 | 8.0 | 1.2 | 1.6 | 6.0 | 8.0 |
| glycollate | | | | | | |
| Colloidal silica | 0.6 | 0.6 | 0.9 | 1.2 | 0.45 | 0.6 |
| Mg stearate | 1.0 | 1.0 | 1.5 | 2.0 | 0.75 | 1.0 |
| TOTAL MASS | 200 | 200 | 300 | 400 | 150 | 200 |

Preparation of Capsule Composition:

Risperidone salt is mixed well with 50% of the amount of the microcrystalline cellulose (MCC), then the other 50% of the MCC is added and mixed, followed by mixing with the lactose and the sodium starch glycollate. Finally the silica is added and mixed. The entire blend is screened over a 850 micrometer sieve, and mixed again, then Mg stearate is added and mixed, resulting in a blend for capsule filling. The blend is filled into capsule size no.3 (150 mg, 200 mg), no.1 (300 mg) or no.0 (400 mg) containing a dose of 1 mg to 8 mg of risperidone respectively.

### EXAMPLE 12

### Pharmaceutical Tablets

Composition of the tablet mass (per tablet, in mg)

| | Strength(risperidone base) | | | | | | | |
|---|---|---|---|---|---|---|---|---|
| | 0.25 mg | 0.5 mg | 1 mg | 2 mg | 3 mg | 4 mg | 6 mg | 8 mg |
| Risperidone(as salt) | 0.25 | 0.5 | 1.0 | 2.0 | 3.0 | 4.0 | 6.0 | 8.0 |
| Lactose monohydrate* | 34.75 | 69.5 | 139.0 | 138.0 | 207.0 | 276.0 | 99.0 | 132.0 |
| Microcrystalline | 12.5 | 25.0 | 50.0 | 50.0 | 75.0 | 100.0 | 37.5 | 50.0 |
| cellulose | | | | | | | | |
| Sodium starch glycollate | 2.0 | 4.0 | 8.0 | 8.0 | 12.0 | 16.0 | 6.0 | 8.0 |
| Mg stearate | 0.5 | 1.0 | 2.0 | 2.0 | 3.0 | 4.0 | 1.5 | 2.0 |
| TOTAL MASS | 50 | 100 | 200 | 200 | 300 | 400 | 150 | 200 |

Coating

| | | | | | | | | |
|---|---|---|---|---|---|---|---|---|
| Opadry | 5.0% | 3.25% | 2.0% | 2.0% | 1.83% | 1.65% | 2.25% | 2% |
| II | | | | | | | | |

Preparation:

Risperidone salt is mixed well with 40% of the MCC (=10% tablet weight), then the other 60% of the MCC, and 30% of the lactose is added and mixed. The remaining lactose and the sodium starch glycollate are mixed, then Mg stearate is added and the blend is mixed, resulting in a blend for compression. The blend is compressed into tablets containing 1-8 mg of risperidone. The compressed tablets may be coated with the coating composition.

### EXAMPLE 13

### Oral Solution Containing Sorbitol

Three different salts of risperidone were added into three oral solutions:

Composition (per 1 g):

| | |
|---|---|
| Risperidone dihydrochloride: | 1.18 mg (eq to 1.0 mg of the base) |
| Risperidone hemimalate: | 1.16 mg (eq to 1.0 mg of the base) |
| Risperidone hydrogen maleate: | 1.28 mg (eq to 1.0 mg of the base) |

The remaining excipients were the same in each solution:

Other excipients:

| | |
|---|---|
| benzoic acid | 2 mg |
| Sorbitol | 20 mg (2%) |
| water | q.s. |
| Total | 1000 mg |

Preparation:

Dissolve the benzoic acid in 60% of the water (heat if necessary to 90° C., allow to cool afterwards). Dissolve the risperidone salt in this solution upon stirring. The sorbitol is dissolved in the remaining water (40%). Combine both solutions and make up the volume with water.

### EXAMPLE 14

### Sample Protocol for Determination of the Solubility in Water

A saturated solution of the risperidone salt in water was prepared by stirring 100-500 mg in 5 ml of water in a thermostated bath (20°±1° C.). Then, 2 ml of the suspension was filtered over a micropore filter. Finally, 1.0 ml of the filtrate was freeze-dried, followed by weight determination of the residue. The maximum amount of the salt dissolved (recalculated to the base), was calculated from this weight determination.

### EXAMPLE 15

### Hygroscopicity Test

The salts were subjected to storage at normal conditions (open air) and at 40° C./75% relative humidity. Water content was determined by Karl Fischer titration. The results are shown in the table.

Water content
in % at

| | | 2 weeks, | 2 weeks, |
|---|---|---|---|
| Salt | t = 0 | normal | 40/75 |
| Dihydrochloride | 0.13 | 0.15 | 0.15 |
| Mesylate | 0.41 | 1.38 | 8.51 |
| Hemitartrate | 0.28 | 0.56 | 0.59 |
| Acetate hydrate | 3.78 | 3.39 | 3.66 |
| Hydrogenmaleate | 0.09 | 0.09 | 0.07 |
| Hemimalate | 0.89 | 0.95 | 1.09 |

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. A salt of risperidone in solid state having a water solubility of at least 10 mg/ml.

2. The salt according to claim 1, having a water solubility of at least 20 mg/ml.

3. The salt according to claim 2, having a water solubility within the range of 20 to 200 mg/ml.

4. The salt according to claim 1, wherein said salt is a pharmaceutically acceptable acid addition salt of risperidone.

5. The salt according to claim 4, wherein said salt is an acid addition salt of risperidone with an acid selected from the group consisting of hydrochloric acid, methane sulfonic acid, benzene sulfonic acid, tartaric acid, maleic acid, malic acid, ethane disulfonic acid, lactic acid, acetic acid, and mandelic acid.

6. The salt according to claim 4, wherein said salt is selected from the group consisting of risperidone dihydrochloride, risperidone mesylate, risperidone hemitartrate, risperidone hydrogenmaleate, risperidone (L)-hemimalate, risperidone hemiedisylate, risperidone (L)-lactate, risperidone acetate monohydrate, and risperidone (R)-mandelate.

7. The salt according to claim 1, wherein said salt is in substantially isolated form.

8. The salt according to claim 7, wherein said salt is at least 99% pure.

9. The salt according to claim 1, wherein said salt is at least 99% pure.

10. A pharmaceutical composition comprising the salt according to claim 1 and at least one pharmaceutically acceptable excipient.

11. A process for making a solid state water soluble salt of risperidone, which comprises:
contacting a risperidone donor with a suitable acid in an organic solvent to form a water soluble risperidone salt; and
precipitating said risperidone salt from said solvent.

12. The process according to claim 11, wherein said solvent is selected from the group consisting of an alcohol or an ester.

13. The process according to claim 12, wherein said solvent is methanol or ethanol.

14. The process according to claim 11, wherein said salt is selected from the group consisting of risperidone dihydrochloride, risperidone mesylate, risperidone hemitartrate, risperidone hydrogenmaleate, risperidone (L)-hemimalate, risperidone hemiedisylate, risperidone (L)-lactate, risperidone acetate monohydrate, and risperidone (R)-mandelate.

15. A salt of risperidone selected from the group consisting of risperidone dihydrochloride, risperidone hydrogenmaleate, risperidone hemitartrate and risperidone hemimalate.

16. The salt according to claim 17, wherein said salt is in solid state.

17. The salt according to claim 17, wherein said salt is in dissolved form.

18. A pharmaceutical composition, comprising a salt according to claim 17 and a pharmaceutically acceptable excipient.

19. The pharmaceutical composition according to claim 20, wherein said composition is an aqueous liquid or suspension.

20. The pharmaceutical composition according to claim 21, wherein said composition is a solid dosage form.

21. A method for treating psychotic disorders in a mammal, which comprises administering an effective amount of a risperidone salt according to claim 1 to a mammal in need thereof.

22. A method for treating psychotic disorders in a mammal, which comprises administering an effective amount of a risperidone salt according to claim 17 to a mammal in need thereof.
